(19) United States
(12) Patent Application Publication
(10) Pub. No.: US 2025/0040923 A1
(43) Pub. Date: Feb. 6, 2025

(54) FLEXIBLE ROBOTIC SURGICAL SYSTEM HAVING ACTUATABLE SUTURING TOOL

(71) Applicant: Boston Scientific Scimed, Inc., Maple Grove, MN (US)

(72) Inventors: John Mims, Austin, TX (US); Luis Amador, JR., Pflugerville, TX (US); Jason N. Mehta, Austin, TX (US); Thomas Neudeck, Austin, TX (US); Michael Fries, Kingsland, TX (US); Vladimir Mitelberg, Austin, TX (US); Andrew MacLeod, Cambridge (GB); Douglas Ivan Jennings, Hertfordshire (GB)

(73) Assignee: Boston Scientific Scimed, Inc., Maple Grove, MN (US)

(21) Appl. No.: 18/793,101

(22) Filed: Aug. 2, 2024

Related U.S. Application Data
(60) Provisional application No. 63/530,692, filed on Aug. 3, 2023.

(51) Int. Cl. A61B 17/04; A61B 17/06; A61B 34/30

(52) U.S. Cl. CPC A61B 17/0469; A61B 17/06066; A61B 34/30; A61B 2017/0496; A61B 2017/06052; A61B 2034/301; A61B 2034/303

(57) ABSTRACT

A robotic system has a flexible portion adapted for insertion into the gastrointestinal tract and includes a suturing system with a needle holder. An actuator is coupled to the needle holder and is adapted to act on the needle holder arm and needle such that the needle can be repeatedly moved through a path. The actuator is driven by a drive mechanism from the robotic system.

### CROSS-REFERENCE TO RELATED APPLICATIONS

This application claims the benefit of priority under 35 U.S.C. § 119 to U.S. Provisional Application No. 63/530,692, filed Aug. 3, 2023, the entire disclosure of which is hereby incorporated by reference herein for all purposes.

### BACKGROUND

### 1. Field

The present disclosure relates to robotic treatment systems for treating a mammalian body. More particularly, this disclosure relates to flexible robotic systems with actuatable tools, and systems and methods for actuating the tools.

### 2. State of the Art

The condition of obesity means an individual has too much body fat and also that an individual's weight is higher than what is considered to be healthy for their height. Biology plays a big role in why some people become obese, but not getting enough exercise, eating more food than the body can use, and drinking too much alcohol also contributes to people becoming obese. Obesity is a major health threat because excess weight puts more stress on every part of the body and puts people at risk of several health problems, such as diabetes, heart disease, and stroke.

For some people, lifestyle changes like maintaining a healthy diet and exercising regularly can help them drop body fat and stop being obese. For others though, it can be extremely difficult to lose body fat and consistently maintain weight loss. Medications for losing weight are available on the market, but some can have serious side effects and may not actually be effective. For obese individuals who cannot lower their amount of body fat through lifestyle changes or medications, various surgical options have become available.

Gastric bypass surgery was the first commonly practiced procedure performed to make the stomach smaller. The procedure involves stapling portions of the stomach wall together and then relocating a small part of the small intestine to the newly formed stomach pouch. By reducing the size of the stomach, the stomach holds less food, the individual obtains a sensation of fullness quicker, fewer calories are eaten, fewer calories are absorbed, and weight loss results. However, there are downsides to the procedure. The procedure is an open surgical procedure which has its own risks, including the potential for complications and infection, and can have an extensive post-surgical recovery period. The procedure is also relatively complicated requiring a reconfiguration of the small intestines. Also, over time the staples can release allowing the stomach to re-enlarge, rendering the procedure less effective.

Another procedure is the ‘gastric banding’ procedure, primarily with the LAP-BAND® system, in which an inflatable band is inserted through the abdomen and about the stomach in a laparoscopic procedure. The band is wrapped around the upper part of the stomach to form a stoma, or ring. Attached to the ring is a thin tube leading to an access port that is implanted under the skin. A balloon attached to the band contacts the stomach and can be inflated (or deflated) with saline via the access port using a needle. Adding saline tightens the stoma about the stomach to cause an earlier sensation of satiety. If the band is too tight, saline can be withdrawn. An advantage of the gastric banding is that it can be performed in a minimally invasive manner with small laparoscopic incisions into the abdomen with consequent reduced recovery time, and that no reconfiguration of the small intestines is required. Nevertheless, the procedure still requires incisions, infection can result, and the recovery can be uncomfortable. In addition, the patient is left with a permanent port just under their skin which can be undesirable to some.

These types of procedures, when all goes well, can be effective, but, as stated, come with the risks associated with open or laparoscopic surgery, and for that reason they are only prescribed in cases of extreme obesity.

Incisionless fully endoscopic methods of reducing the capacity of the stomach have been developed to surgically treat obesity. Broadly, such methods endoluminally approximate tissue at a portion of the stomach, including at least a portion of the greater curvature of the stomach. The method includes making a pattern of endoscopic stitches in which a significant portion of the stomach is closed off. The resulting stomach reduction procedure can provide a seventy to seventy-five percent reduction in available stomach volume. Because the procedure is incisionless, it is safer to patients and offers an easier recovery.

While the procedure is shown to be an effective method to reduce the capacity of the stomach, create an earlier sensation of satiety, and effect weight loss in an obese patient, there remain obstacles to its application. Bariatric procedures have conventionally been practiced by bariatric surgeons who approach stomach reduction from outside the stomach and are not as familiar with surgery on gastrointestinal structures when viewed from inside the gastrointestinal tract. Meanwhile, gastrointestinal surgeons who are more familiar operating on the stomach from the interior do not have the familiarity with bariatric procedures and as a consequence have a reduced comfort level with such surgeries.

Robotic systems have been used in various surgical procedures, but are not widely used in surgery in the gastrointestinal space. Robotic systems often require instrument that are different than conventional rigid and large robotic instruments to access the gastrointestinal space. Various challenges therefore arise in connection with actuating the end effector at the distal end of the robotic surgical tools.

### SUMMARY

A robotic system is provided having a flexible portion adapted for insertion into the gastrointestinal tract. The flexible portion has a proximal end, a distal end, and a longitudinal axis extending between the proximal and distal ends. The distal end of the flexible portion may have as a generally circular cross-sectional shape. The distal end is provided with a tissue fastening system, preferably in the form of a suturing system. The distal end of the robotic system and the suturing system are suitable for passage through a natural orifice of the patient and into the gastrointestinal tract, and particularly through the mouth, through the esophagus, and into the stomach.

The suturing system may include a needle holder adapted to pass a needle through tissue. A suture needle with suture is preferably removably coupled to the needle holder. An actuator is coupled to the needle holder and is adapted to act on the needle holder arm and needle such that the needle can be moved through a path.

In an embodiment, the needle holder rotates about an axis oriented transverse to the longitudinal axis. The needle holder and actuator are configured such when the actuator acts upon the needle holder, the needle moves in a plane from a first position to a second position. The needle in the second position is preferably oriented parallel to the longitudinal axis of the distal end of the flexible portion of the robotic system.

In embodiments, the needle holder moves linearly. In embodiments, the needle holder moves transverse to the longitudinal axis.

In embodiments, the two needle holders are provided, and the needle is passed from one needle holder to the other.

In embodiments, the needle holder and actuator are configured such that when the actuated acts upon the needle holder, the needle holder and needle are rotated about an axis oriented parallel to the longitudinal axis. In an embodiment, the needle holder and needle are rotated about an axis oriented coaxial with the longitudinal axis.

In embodiments, the needle holder and actuator are configured such when the actuator acts upon the needle holder, the needle holder and needle are moved in a plane parallel with a cross-section through the distal end of the flexible portion of the robotic system. The needle moves from a first position to a second position. The needle in the first position is oriented tangential to a circle extending about the longitudinal axis of the distal end of the flexible portion of the robotic system.

In embodiments, the needle holder and actuator are configured such when the actuator acts upon the needle holder, the needle is moved in a plane oriented transverse to the longitudinal axis of the distal end of the flexible portion of the robotic system.

In embodiment, the needle holder is advanced in a helical pathway.

In embodiments, the actuator rotates about an axis to move the needle holder. In embodiments, the actuator applies a longitudinal tensile force to move the needle holder. In embodiments, the actuator applies a longitudinal compressive force to move the needle holder. In embodiments, the actuator is coupled to gears or a toothed rack. In embodiments, the actuator is coupled to belts. In embodiment, the actuator reciprocates a shaft.

The suturing system may include a tissue grasper adapted to engage tissue and retract the engaged tissue into the path of the needle so that the moved needle extends through the retracted tissue. In embodiments, the tissue grasper may extend within a lumen of the flexible portion of the robotic system. The lumen may be axial or non-axial with the longitudinal axis of the flexible portion of the robotic system.

The suturing system may include a needle capture mechanism to retain and permit removal of the needle from the needle holder when the needle is at an end of the path.

In accordance with embodiments disclosed herein, various actuation systems are provided for actuating the needle holder and advancing the needle through tissue.

In accordance with various principles of the present disclosure, a surgical system for use on a patient includes a flexible robotic instrument having a proximal end and a distal end, the distal end for insertion through a natural orifice of the patient to access a gastrointestinal tract of the patient; and a fastening tool at the distal end, the fastening tool having a first fastener holder arm adapted to move a releasable tissue fastener through a path. In some aspects, the robotic instrument includes an actuator operably coupled to the fastener holder arm to move the first fastener holder arm, and the robotic instrument is adapted to drive the actuator to move the releasable tissue fastener from a first position at a first end of the path to a second position at a second end of the path.

In some aspects, the releasable tissue fastener is a suture needle coupled with a length of suture.

In some aspects, the actuator rotates the first fastener holder arm about an axis.

In some aspects, the actuator includes a flexible transmission member which is driven by being pulled into tension. In some aspects, a spring is adapted to provide a counter force to the flexible transmission member.

In some aspects, the actuator includes a rotatable shaft. In some aspects, at least one of a worm gear, a spline gear, or a bevel gear is provided at an end of the actuator.

In some aspects, the actuator includes a reciprocable shaft. In some aspects, the fastener holding arm is provided with a crank. In some aspects, a rack and pinion coupling is provided between the actuator and the fastener holding arm.

In some aspects, the actuator includes a belt. In some aspects, the actuator is coupled to the fastener holding arm with a constant velocity joint.

In some aspects, the fastening tool has a longitudinal axis, and the first fastener holder arm is rotated in a plane about an axis oriented transverse to the longitudinal axis.

In some aspects, the fastening tool has a longitudinal axis, and the first fastener holder arm is rotated about a rotation axis parallel to the longitudinal axis. In some aspects, the rotation axis is coaxial with the longitudinal axis. In some aspects, the first fastener holder arm is rotated in a plane. In some aspects, the first fastener holder arm is rotated in a helical pathway.

In some aspects, the first fastener holder arm is moved linearly.

In some aspects, the tool includes a second fastener holder arm that cooperates with the first fastener holder arm to pass the tissue fastener therebetween. In some aspects, the first and second fastener holder arm move linear relative to each other. In some aspects, the first and second fastener holder arm move along an arc relative to each other.

In some aspects, the tool includes a track, and the first holder arm is driven in the track by the actuator. In some aspects, the track is partially circular. In some aspects, the track is helical.

In some aspects, the system further a magazine includes of tissue fasteners, wherein a tissue fastener loaded on the first holder arm is deployed, a subsequent tissue fastener from the magazine can be loaded onto the first holder arm.

In some aspects, the system further includes a yoke adapted to engage and temporarily secure the tissue fastener, wherein when the tissue fastener is secured in the yoke, the first holder arm can be decoupled from the tissue fastener. In some aspects, the yoke is operably coupled to the robotic instrument and driven to engage and release the tissue fastener by the robotic instrument.

In some aspects, the fastening tool is provided with a tissue grasper adapted to engage tissue and retract the engaged tissue into the path. In some aspects, the tissue grasper is integrated with the fastening tool. In some aspects, the tissue grasper is mechanically engaged with the fastener holder arm. In some aspects, the robotic instrument is coupled to the tissue grasper such that movement of the tissue grasper results in movement of the fastener holder arm. In some aspects, movement of the fastener holder arm results in movement of the tissue grasper. In some aspects, the tissue grasper and fastener holder arm are mechanically engaged via a gear link. In some aspects, the gear link includes a worm gear. In some aspects, the tissue grasper is operably engaged with the flexible robotic instrument with a pin and slot coupling. In some aspects, the pin and slot coupling is a bayonet connection.

In accordance with various principles of the present disclosure, a tool for a surgical system for use on a patient, includes a base for coupling to a robotic surgical system; a fastener holder arm coupled to the base and adapted to move a releasable tissue fastener through a path; and a tissue grasper adapted to advance relative to the fastener holder arm, grasp tissue, and retract tissue into the path. In some aspects, the fastener holder arm and the tissue grasper operably engaged such that, at times, movement of one of the tissue grasper and the fastener holder arm causes movement of the other of the tissue grasper and the fastener holder arm.

In some aspects, the tissue grasper and fastener holder arm are engaged via a gear link. In some aspects, the gear link includes a worm gear.

### DETAILED DESCRIPTION OF THE DRAWINGS

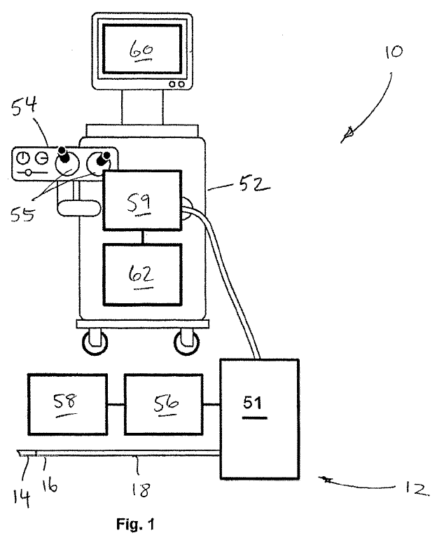

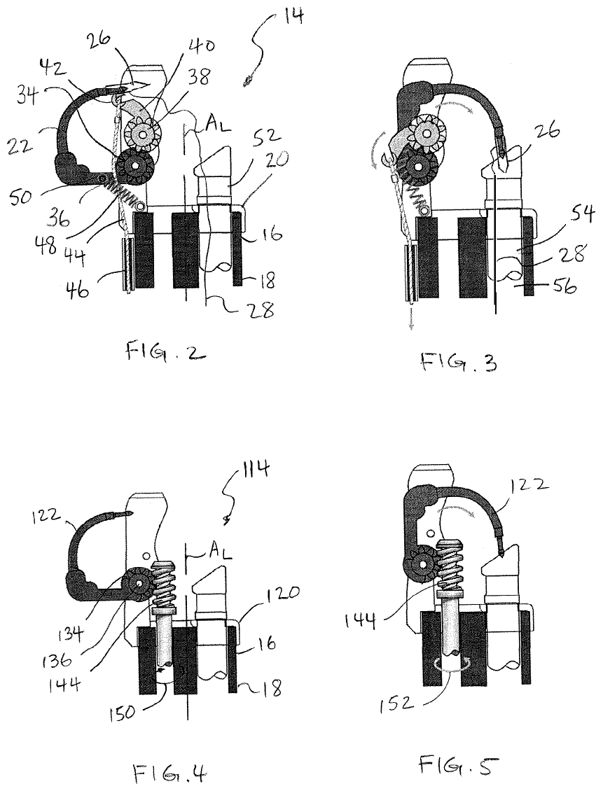

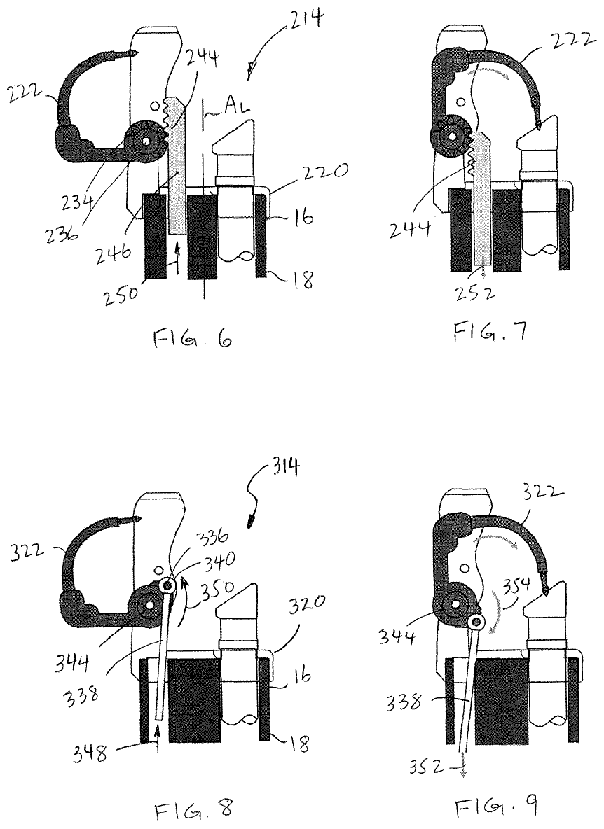

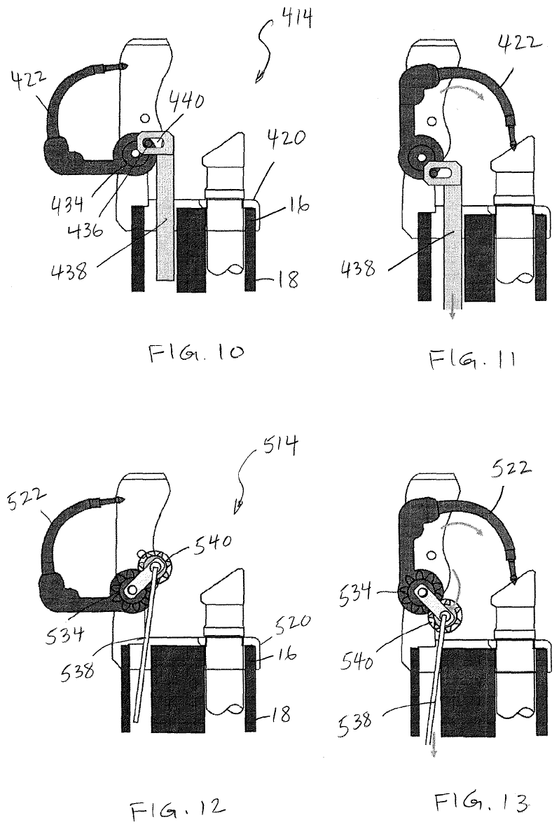

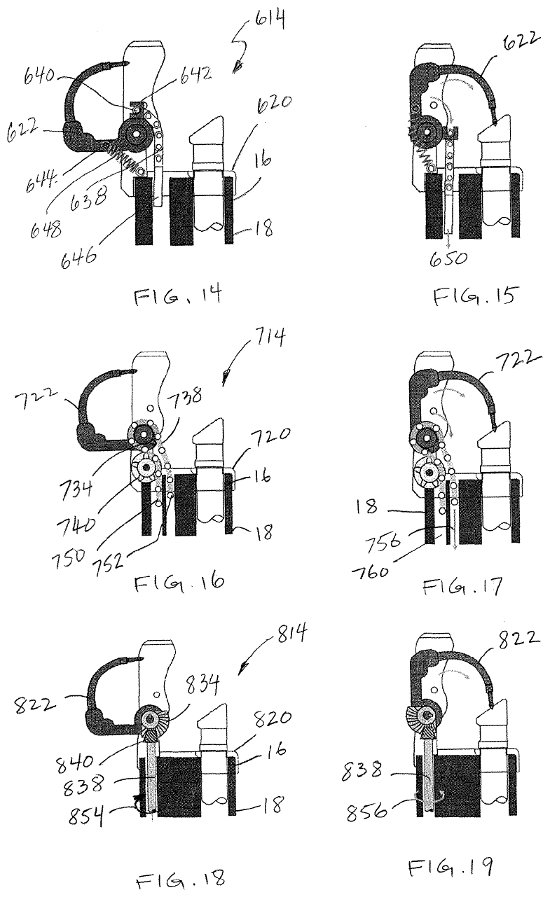

FIG. 1 is a general diagram of a robotic surgical system.

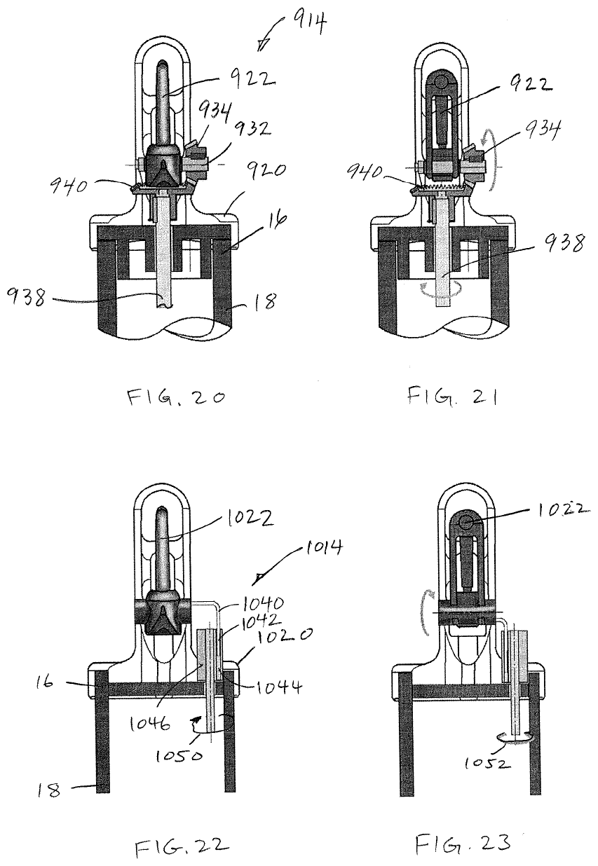

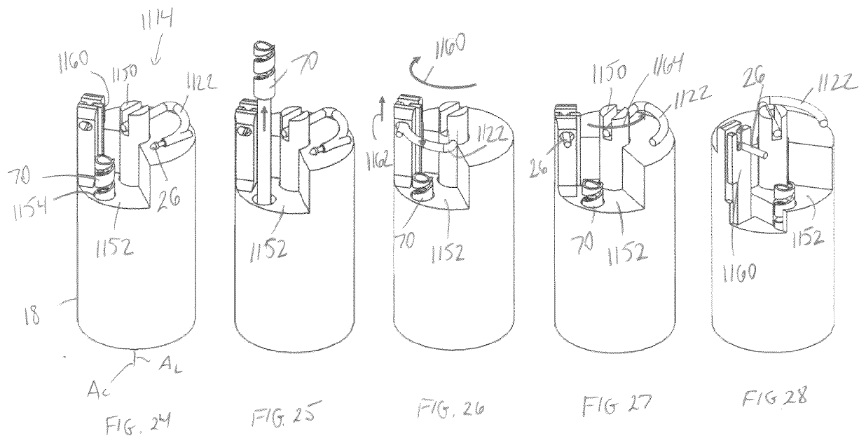

FIG. 2 is a longitudinal section view of the distal end of a first embodiment of a suturing tool module attached to a distal end of a robotic surgical system with a needle holder for holding a needle, with the needle holder in an open first position.

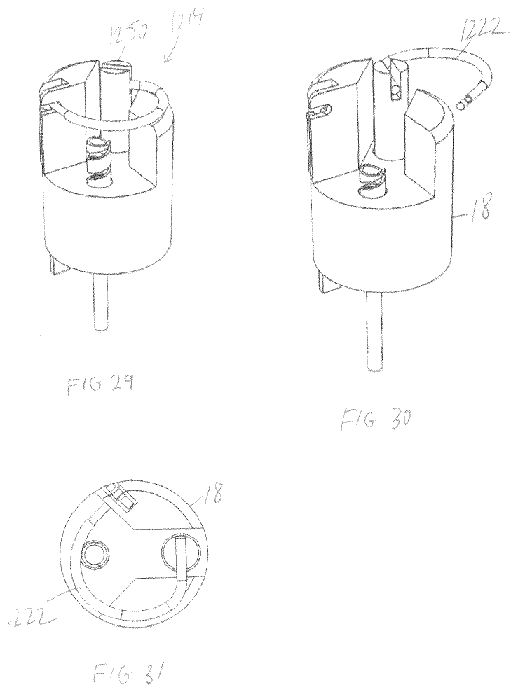

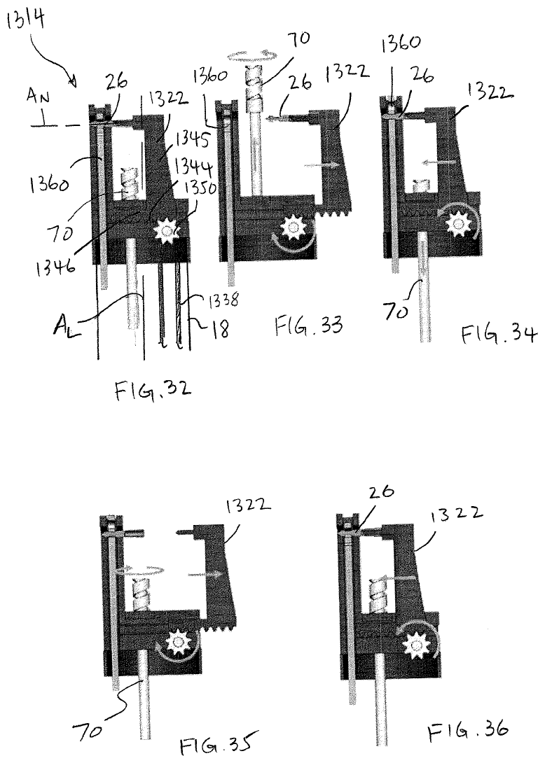

FIG. 3 is a view of the first embodiment similar to FIG. 2, with the needle holder in a closed second position.

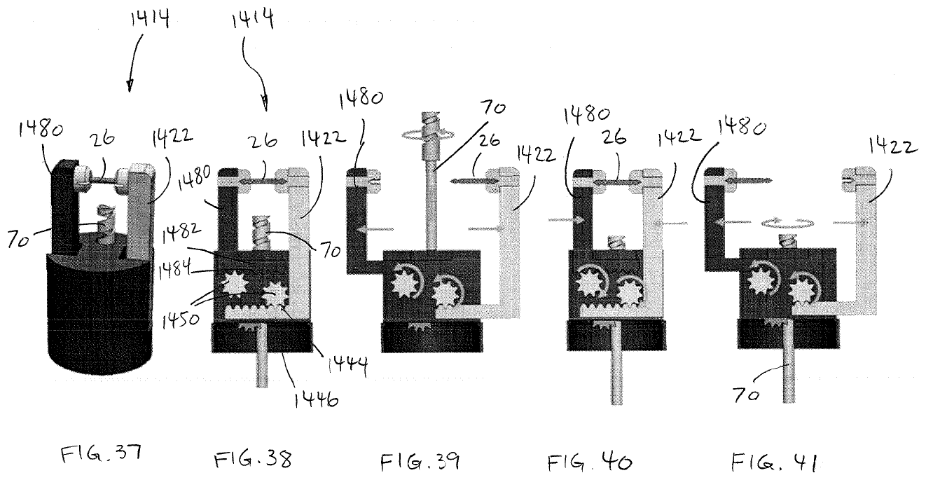

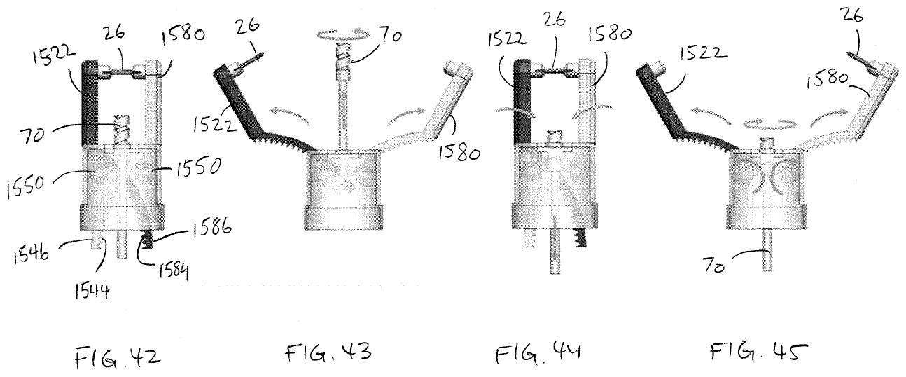

FIG. 4 is a longitudinal section view of the distal end of a second embodiment of a suturing tool module attached to a distal end of a robotic surgical system with a needle holder for holding a needle, with the needle holder in an open first position.

FIG. 5 is a view of the second embodiment similar to FIG. 4, with the needle holder in a closed second position.

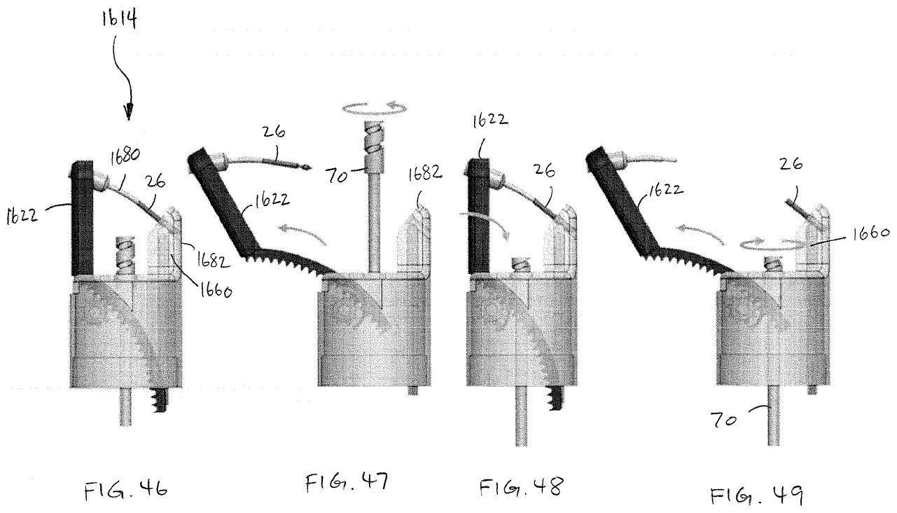

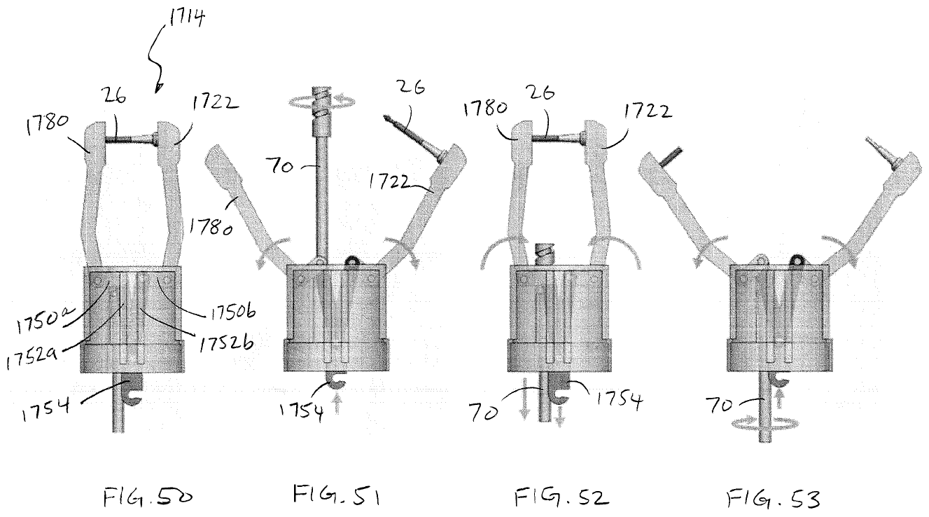

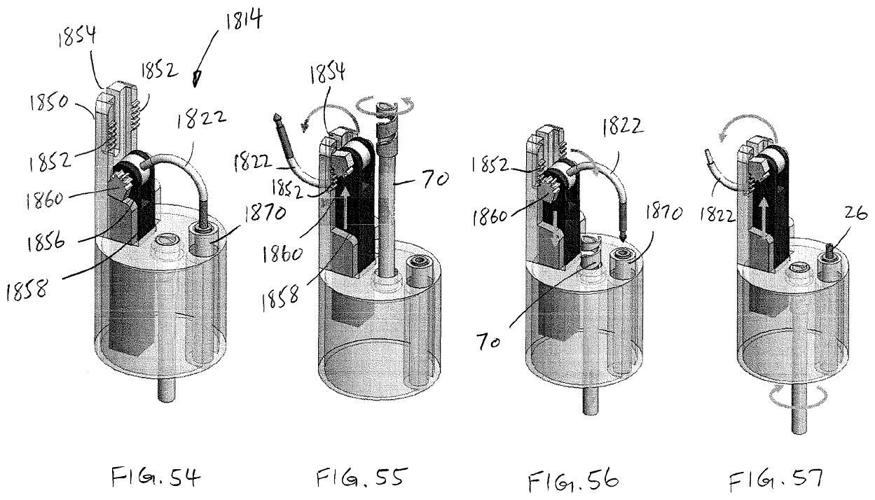

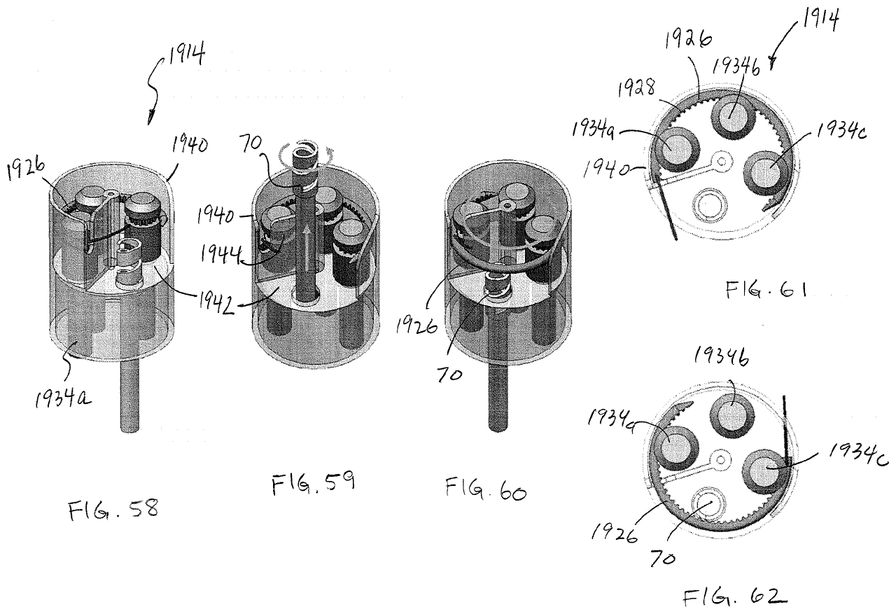

FIG. 6 is a longitudinal section view of the distal end of a third embodiment of a suturing tool module attached to a distal end of a robotic surgical system with a needle holder for holding a needle, with the needle holder in an open first position.

FIG. 7 is a view of the third embodiment similar to FIG. 6, with the needle holder in a closed second position.

FIG. 8 is a longitudinal section view of the distal end of a fourth embodiment of a suturing tool module attached to a distal end of a robotic surgical system with a needle holder for holding a needle, with the needle holder in an open first position.

FIG. 9 is a view of the fourth embodiment similar to FIG. 8, with the needle holder in a closed second position.

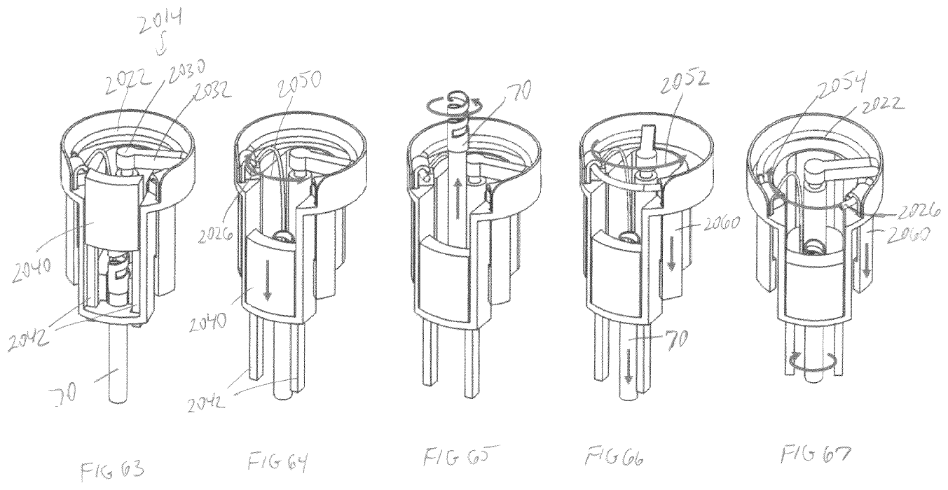

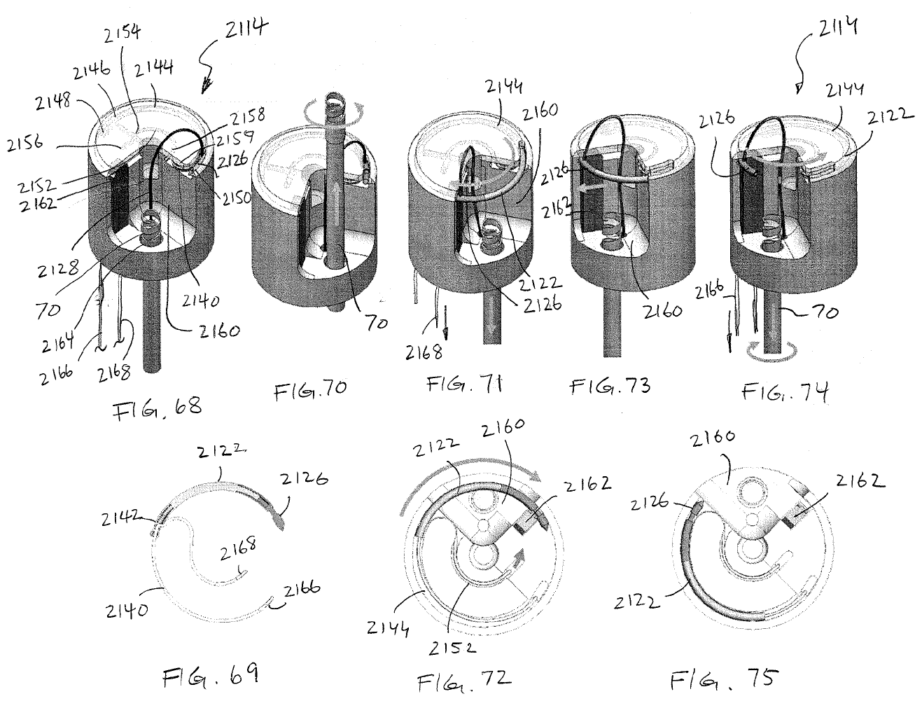

FIG. 10 is a longitudinal section view of the distal end of a fifth embodiment of a suturing tool module attached to a distal end of a robotic surgical system with a needle holder for holding a needle, with the needle holder in an open first position.

FIG. 11 is a view of the fifth embodiment similar to FIG. 10, with the needle holder in a closed second position.

FIG. 12 is a longitudinal section view of the distal end of a sixth embodiment of a suturing tool module attached to a distal end of a robotic surgical system with a needle holder for holding a needle, with the needle holder in an open first position.

FIG. 13 is a view of the sixth embodiment similar to FIG. 12 with the needle holder in a closed second position.

FIG. 14 is a longitudinal section view of the distal end of a seventh embodiment of a suturing tool module attached to a distal end of a robotic surgical system with a needle holder for holding a needle, with the needle holder in an open first position.

FIG. 15 is a view of the seventh embodiment similar to FIG. 14, with the needle holder in a closed second position.

FIG. 16 is a longitudinal section view of the distal end of an eighth embodiment of a suturing tool module attached to a distal end of a robotic surgical system with a needle holder for holding a needle, with the needle holder in an open first position.

FIG. 17 is a view of the eighth embodiment similar to FIG. 16, with the needle holder in a closed second position.

FIG. 18 is a longitudinal section view of the distal end of a ninth embodiment of a suturing tool module attached to a distal end of a robotic surgical system with a needle holder for holding a needle, with the needle holder in an open first position.

FIG. 19 is a view of the ninth embodiment similar to FIG. 18, with the needle holder in a closed second position.

FIG. 20 is a front facing longitudinal section view of the distal end of a tenth embodiment of a suturing tool module attached to a distal end of a robotic surgical system with a needle holder for holding a needle, with the needle holder in an open first position.

FIG. 21 is a view of the tenth embodiment similar to FIG. 20, with the needle holder in a closed second position.

FIG. 22 is a front facing longitudinal section view of the distal end of an eleventh embodiment of a suturing tool module attached to a distal end of a robotic surgical system with a needle holder for holding a needle, with the needle holder in an open first position.

FIG. 23 is a view of the eleventh embodiment similar to FIG. 22, with the needle holder in a closed second position.

FIG. 24 is a perspective distal end view of a twelfth embodiment of a suturing tool module for attachment to a distal end of a robotic surgical system with a needle holder for holding a needle, with the needle holder in an open first position.

FIGS. 25 through 28 illustrate actuation and operation of the twelfth embodiment of the suturing tool module to move the needle holder and needle.

FIGS. 29 and 30 are perspective distal end views of a thirteenth embodiment of a suturing tool module for attachment to a distal end of a robotic surgical system with a needle holder for a holding a needle, the holder shown in a closed first position (FIG. 29) and an open second position (FIG. 30).

FIG. 31 is an end view of the thirteenth embodiment of a suturing tool module, with the holder shown in the first closed position.

FIGS. 32 through 36 are schematic longitudinal section views of the distal end of a fourteenth embodiment of a suturing tool module for a robotic surgical system with a needle holder for holding a needle, illustrating actuation and operation of the suture tool module.

FIG. 37 is a perspective view of a distal end of a fifteenth embodiment of a suturing tool module for a robotic surgical system with two needle holders for holding a needle.

FIGS. 38 through 41 are schematic longitudinal section views of the distal end of the fifteenth embodiment of a suturing tool module for a robotic surgical system with a needle holder for holding a needle, illustrating actuation and operation of the suture tool module.

FIGS. 42 through 45 are partially transparent views of the distal end of a sixteenth embodiment of a suturing tool module for a robotic surgical system with two needle holders for holding a needle, illustrating actuation and operation of the suture tool module.

FIGS. 46 through 49 are partially transparent views of the distal end of a seventeenth embodiment of a suturing tool module for a robotic surgical system with a needle holder for holding a needle, illustrating actuation and operation of the suture tool module.

FIGS. 50 through 53 are partially transparent views of the distal end of an eighteenth embodiment of a suturing tool module for a robotic surgical system with two needle holders for holding a needle, illustrating actuation and operation of the suture tool module.

FIGS. 54 through 57 are partially transparent perspective views of the distal end of a nineteenth embodiment of a suturing tool module for a robotic surgical system with a needle holder for holding a needle, illustrating actuation and operation of the suture tool module.

FIGS. 58 through 60 are partially transparent perspective views of a distal end of a twentieth embodiment of a suturing tool module for a robotic surgical system with a needle holder for holding a needle, illustrating actuation and operation of the suture tool module.

FIG. 61 is a distal end view of the embodiment and configuration shown in FIG. 59, and FIG. 62 is a distal end view of the embodiment and configuration shown in FIG. 60.

FIGS. 63 through 67 are partially transparent perspective views of a distal end of a twenty-first embodiment of a suturing tool module for a robotic surgical system with a needle holder for holding a needle and a magazine of needles, illustrating actuation and operation of the suture tool module.

FIGS. 68 through 75 illustrate a twenty-second embodiment of a suturing tool module for a robotic surgical system with a needle holder for holding a needle.

FIGS. 68, 70, 71, 73 and 74 are perspective views illustrating actuation and operation of the suture tool module.

FIG. 69 illustrates the needle holder, needle, and path of the actuation wire.

FIG. 72 is a distal end view of the embodiment and configuration shown in FIG. 71.

FIG. 75 is a distal end view of the embodiment and configuration shown in FIG. 68.

FIGS. 76 through 80 are partially transparent perspective views of a distal end of a twenty-third embodiment of a suturing tool module for a robotic surgical system with a helical needle holder for holding a needle, illustrating actuation and operation of the suture tool module.

FIGS. 81 through 87 are partially transparent perspective views of a distal end of a twenty-fourth embodiment of a suturing tool module for a robotic surgical system with a needle holder and tissue grasper mechanically engaged such that, at times, movement of one results in movement of the other, illustrating actuation and operation of the suture tool module.

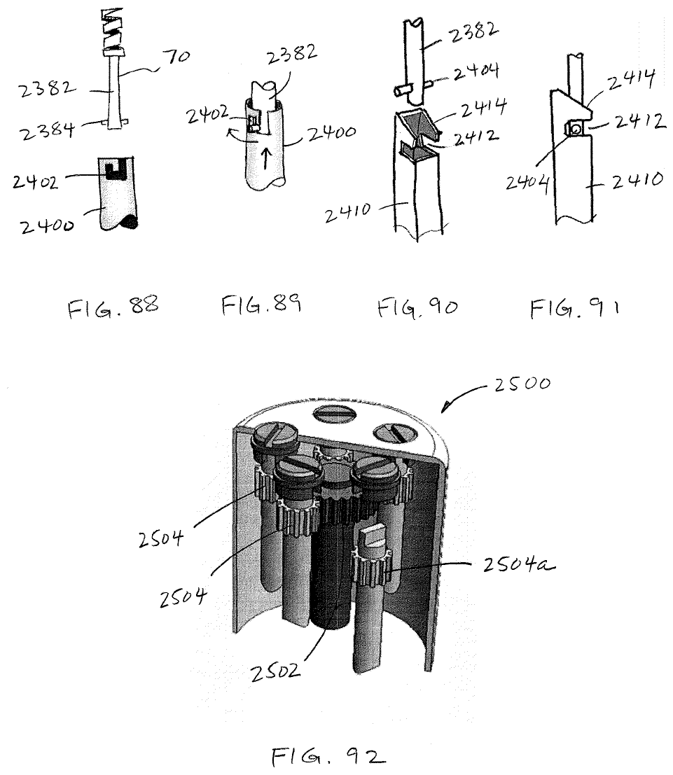

FIGS. 88 and 89 are schematic illustrations of one coupling of a drive shaft of the robotic system to a tool on the suturing tool module.

FIGS. 90 and 91 are schematic illustrations of another coupling of a drive shaft of the robotic system to a tool on the suturing tool module.

FIG. 92 is a partial cutaway view of a planetary drive system for driving tools of the suturing tool module.

### DETAILED DESCRIPTION

Various devices, structures, and methods for coupling a robotic system with a system for affecting tissue are described herein with reference to various examples of embodiments illustrated in the accompanying drawings. In accordance with various principles of the present disclosure, a robotic system is configured to be coupled with a system configured to affect tissue of a patient, such as to associate a device with respect to the tissue. In some aspects, the device to be associated with tissue is a suture, a suture anchor, an anchor, or the like. The robotic system and/or the system affecting tissue are movable longitudinally/axially (e.g., longitudinally and/or axially translatable) about a longitudinal axis thereof, and/or rotatable about such longitudinal axis. In accordance with various principles of the present disclosure, at least a portion of the robotic system, such as at least a portion of the distal end of the robotic system, is configured to axially translate and/or rotate at least a component of the system affecting tissue to actuate such system to deliver and/or deploy and/or manipulate a device with respect to tissue. In some aspects, the robotic system includes a robotic arm, the robotic arm having a distal end, the distal end of the robotic arm forming a distal end of the system couplable with the system configured to affect tissue.

Turning now to FIG. 1, in one example of an embodiment of a robotic system 10 formed in accordance with various principles of the present disclosure, the system 10 includes an endoluminal robot 12 having a proximal end and a distal end. A suturing tool 14 is provided at the distal end 16 of the robot 12. The endoluminal robot 12 includes a robotic, shapeable insertion tube 18 sized in diameter and length to be advanced into and through a natural orifice of a patient and to a target location in the gastrointestinal tract. By way of example, the insertion tube is sized in diameter and length to be inserted into and through the mouth and esophagus of human patient, and to extend from outside the patient to a target location in the stomach. The robotic system 12 is aware of the shape of the insertion tube and the location of the insertion tube in space. In a preferred embodiment, the suturing tool 14 is in the form of a tool module removably attached to the distal end 16 of the robotic system 12 for cleaning, sterilization, disposal and/or replacement. Nevertheless, a suturing tool or any portion or tool thereof may be permanently integrated at the distal end of the endoluminal robot.

The robotic system 12 includes a mechanized system 51 to control the movement of the insertion tube 18 and to operate the suturing tool 14. More particularly, the mechanized system 51 may include actuators, such as push-pull actuators, rotatable drivers, and/or gear drive mechanisms 51 to effect movement of the components operating at and through the suturing tool 14. The mechanized system 51 may include a control system 52 to provide input to mechanized system 51 based on inputs from the surgical system. The inputs can include human interface controls 54 and feedback from first sensors 56. The human interface controls 54 converts human manual input to movement of the insertion tube 18 via the mechanized system 51. The human interface controls 54 can include joysticks 55, trackballs, a keyboard, buttons, knobs, haptic gloves, and/or any other suitable interface to permit input from an operator. The first sensors 56 can include load cells and strain gauges coupled to the actuators in the mechanized system 51 to monitor forces applied by the mechanized system and actuators. First sensors 56 may be located within the insertion tube or coupled via mechanical, optical, or electrical components to sensors external of the insertion tube. Other sensors 59 may be provided to sense and identify the patient environment and optionally the needle and/or the suture. The robotic surgical system also includes a light source and camera 58, and a visual display 60 to display images from the camera 58 optionally augmented by input from the sensors and/or patient data. The robotic surgical system also includes a processing system 62 including a microprocessor that runs the robotic system software, a memory for storing software, an interface to access patient data, and which integrates the inputs from the subsystems together to facilitate operation of the insertion tube 18 and suturing tool 14 to perform a surgical procedure.

Turning to FIGS. 2 and 3, a first embodiment of a suturing tool 14 includes an end cap 20 adapted for placement at and connection to the distal end 16 of the insertion tube 18. The insertion tube 18 includes a longitudinal axis A_{L}. The end cap 20 includes a needle holder arm 22 mounted to the end cap 20 and which is rotatable to move a removable needle 26 (FIG. 3) provided with a length of suture 28 through patient tissue. The needle holder arm 22 rotates on an axis 32 oriented transverse to the longitudinal axis A_{L}. The needle holder arm 22 is configured to move the needle 26 in an arc within a plane from a first position to a second position. The needle in the second position is oriented substantially parallel to the longitudinal axis of the distal end 16 of the flexible portion of the robotic system.

In the first embodiment, a sprung cable mechanism is provided to effect movement of the needle holder arm 22 and the needle 26. The needle holder arm 22 is rotatably mounted on a mounting bracket 30. The needle holder arm 22 has a circular gear 34 at a mounting end. Circular gear 34 rotates on axis 36 that extends transverse to the longitudinal axis A_{L}. A bell crank 38 is rotatably coupled to the mounting bracket 30 and meshed with the circular gear 34. The gear ratio between the gear 34 and the bell crank 38 can be 1:1; alternatively, the ratio between the gear 34 and the bell crank 38 can be adjusted to obtain a higher tissue penetration force. The bell crank 38 has an arm 40 with a catch 42. A flexible transmission member 44, such as a cable, extends through a lumen 46 of the tube 18 and is coupled to the catch 42 on the arm 40 of the bell crank 38. A tension or torsion spring 48 is coupled to between a portion 50 of the needle holder arm 22 and the end cap 20. The spring 48 operates to bias the needle holder arm 22 into an open (first) position in which the needle 26 is generally transverse to the longitudinal axis A_{L}. When tension is applied to the cable 44, the bell crank 38 is rotated, and the needle holder arm 22 is caused to rotate in an arc-like path into a closed (second) position (FIG. 3). In the closed second position, the needle 26 extends generally parallel to the longitudinal axis A_{L}. In an embodiment, the end cap 20 includes a distally extending tube 52 that is adapted to help guide tissue into a folded shape appropriate for successful suturing, as well as protect the needle, and provide a garage for needle exchange. In the second position, the needle 26 is positioned at least partially within the tube 52. In addition, the tube 52 may include a lower portion 54 that provides protection of a working channel 56 of the insertion tube 18 and/or facilitates engagement between the end cap 20 and the distal end of the insertion tube 18. When tension is released from the cable 44, the spring 48 again operates to apply an opening force to needle holder arm 22 to move the needle 26 back to the first position. As such, the forces at the distal end and simple operation of a flexible transmission member actuate the needle holder arm and consequent movement of the needle 26.

Turning now to FIGS. 4 and 5, a second embodiment of a suturing tool 114 includes an end cap 120 adapted for placement at and connection to the distal end 16 of the insertion tube 18. The insertion tube 18 includes a longitudinal axis A_{L}. The end cap includes a needle holder arm 122 mounted to the end cap 120 and which is rotatable to move a removable needle (not shown) through patient tissue. Suturing tool 114 operates substantially the same as the first embodiment 14 with the exception of the following modifications. The needle holder arm 122 has a circular gear 134 at a mounting end. Circular gear 134 rotates on axis 136 that extends transverse to the longitudinal axis A_{L}. A worm gear 144 extends from a shaft 146 extending from the distal end 16 of the insertion tube 18 and meshes with the circular gear 134. The worm gear 144 is rotatably driven by the mechanized system 51 (FIG. 1). When worm gear 144 is driven fully in a first rotational direction 150, the needle holder arm 122 moves the needle into the first (open) position. When the worm gear 144 is driven in a second rotational direction 152, the needle holder arm 122 moves the needle (not shown) into the second (closed) position.

Turning now to FIGS. 6 and 7, a third embodiment of a suturing tool 214 includes an end cap 220 adapted for placement at and connection to the distal end 16 of the insertion tube 18. The end cap 220 includes a needle holder arm 222 mounted to the end cap 220 and which is rotatable to move a removable needle (not shown) through patient tissue. Suturing tool 214 operates substantially the same as the first embodiment of suturing tool 14 with the exception of the following modifications. The needle holder arm 222 has a circular gear 234 at a mounting end. Circular gear 234 rotates on axis 236 that extends transverse to the longitudinal axis A_{L}. A rack 244 extends from a longitudinally displaceable shaft 246 which translates relative to the distal end 16 of the insertion tube 18. The rack 244 meshes with the circular gear 234. The shaft 246 supporting the rack 244 is longitudinally displaceable by the mechanized system 51 (FIG. 1). When the rack 244 is at a distal first position, the circular gear 234 is driven in a first rotational direction 250 to move the needle holder arm 222 into the first (open) position. Referring to FIG. 7, when the rack 244 is driven into a proximal second position 252, the needle holder arm 222 moves the needle (not shown) into the second (closed) position.

Turning now to FIGS. 8 and 9, a fourth embodiment of a suturing tool 314 includes an end cap 320 adapted for placement at and connection to the distal end 16 of the insertion tube 18. The end cap 320 includes a needle holder arm 322 mounted to the end cap 320 and which is rotatable to move a removable needle (not shown) through patient tissue. Suturing tool 314 operates substantially the same as the first embodiment of suturing tool 14 with the exception of the following modifications. The needle holder arm 322 has a rotatable crank 334 at a mounting end. The rotatable crank 334 includes a pin 336. A connecting rod 338 extends from the distal end 16 of the insertion tube 18 and includes an opening 340 at which the rod 338 connects to the pin 336. Alternatively, the crank 334 may include an opening and the rod 338 may include a pin. The rod 338 is longitudinally displaceable by the mechanized system 51 (FIG. 1). When the rod 338 is moved in a distal first direction 348, the crank 334 is driven in a first rotational direction 350 to move the needle holder arm 322 into the first (open) position. Referring to FIG. 9, when the rod 338 is driven in an opposite second direction 352, the crank 344 is rotated in a second direction 354 to rotate the needle holder arm 322 to move the needle (not shown) into the second (closed) position.

Turning now to FIGS. 10 and 11, a fifth embodiment of a suturing tool 414 includes an end cap 420 adapted for placement at and connection to the distal end 16 of the insertion tube 18. Suturing tool 414 operates substantially the same as the first embodiment of suturing tool 14 with the exception of the following modifications. The needle holder arm 422 is driven from the mechanized system 51 by a crank and yoke system. The needle holder arm 422 has a rotatable crank 434 at a mounting end. The rotatable crank 434 includes a pin 436. A reciprocable yoke 438 extends from the distal end 16 of the insertion tube 18. The distal end of the yoke 438 includes slot 440 in which the pin 436 is engaged. As the yoke 438 is longitudinally displaced, the crank 434 is rotated to operate rotation of the needle holder arm 422. The yoke 438 is longitudinally displaceable by the mechanized system 51 (FIG. 1) to move the needle holder arm between the positions shown in FIGS. 10 and 11.

Turning now to FIGS. 12 and 13, a sixth embodiment of a suturing tool 514 includes an end cap 520 adapted for placement at and connection to the distal end 16 of the insertion tube 18. The end cap 520 includes a needle holder arm 522 mounted to the end cap 520 and which is rotatable to move a removable needle (not shown) through patient tissue. Suturing tool 514 operates substantially the same as the first embodiment of suturing tool 14 with the exception of the following modifications. The needle holder arm 522 is driven from the mechanized system 51 by a planetary gear system. The planetary gear system includes circular ‘sun’ gear 534 fixed to the end of the needle holder arm 522, and a rotatable ‘planet’ gear 540 mounted on the rod 538. The rod 538 extends from the distal end 16 of the insertion tube 18. As the rod 538 is longitudinally displaceable by the mechanized system 51 (FIG. 1), the needle is rotated between the positions shown in FIGS. 12 and 13. When the two gears 534, 540 have the same number of teeth, the needle holder arm 522 and needle can be rotated 90° (between the positions of FIGS. 12 and 13) by a 45° rotation of the planet gear 540 relative to the sun gear 534.

Turning now to FIGS. 14 and 15, a seventh embodiment of a suturing tool 614 includes an end cap 620 adapted for placement at and connection to the distal end 16 of the insertion tube 18. The end cap 620 includes a needle holder arm 622 mounted to the end cap 620 and which is rotatable to move a removable needle (not shown) through patient tissue. Suturing tool 614 operates substantially the same as the first embodiment of suturing tool 14 with the exception of the following modifications. The needle holder arm 622 is driven by the mechanized system 51 by a chain 638 and tension or torsion spring 648. The distal end 640 of the chain 638 is connected to an arm 642 radially extending from a mounting hub 644 on which the needle holder arm 622 rotates. The proximal end of the chain 638 may extend to the mechanized system 51 or be coupled to a rod or cable 646 that is coupled to the mechanized system. Tension 650 applied to the chain 638 by the mechanized system 51 operates to move the needle holder arm 622 from a first (open) position (FIG. 14) to a second (closed) position (FIG. 15). The spring 648 extends between the needle holder arm 622 and the end cap 620. The spring 648 holds the needle holder arm 622 in the first position when overcoming tension 650 is not applied to the chain 638, and then returns the needle holder arm 622 to the first position after the needle holder arm 622 has been moved to the second position and the tension 650 is released from the chain 638 (FIG. 14).

Turning now to FIGS. 16 and 17, an eighth embodiment of a suturing tool 714 includes an end cap 720 adapted for placement at and connection to the distal end 16 of the insertion tube 18. The end cap 720 includes a needle holder arm 722 mounted to the end cap 720 and which is rotatable to move a removable needle (not shown) through patient tissue. Suturing tool 714 operates substantially the same as the first embodiment of suturing tool 14 with the exception of the following modifications. The needle holder arm 722 is driven by a belt drive system. In an embodiment, the belt drive system includes a first cog 734 at a mounting end of the needle holder arm 722, a second cog 740 mounted proximal of the first cog 734, and a drive belt 738 looped over the first cog, extending between the first and second cogs, and engaging the first and second cogs. When a first end 750 of the drive belt 738 is pulled, the needle holder arm 722 is rotated into a first (open) position (FIG. 16). When a second end 752 of the drive belt 738 is pulled in the direction of arrow 756, the needle holder arm 722 is rotated into a second (closed) position (FIG. 17). The first and second ends 750, 752 of the drive belt extend through one or more lumen 760 of the insertion tube 18 and are coupled to the mechanized system 51, which is adapted to pull the ends as necessary for operation of the needle holder arm.

Turning now to FIGS. 18 and 19, a ninth embodiment of a suturing tool 814 includes an end cap 820 adapted for placement at and connection to the distal end 16 of the insertion tube 18. The end cap 820 includes a needle holder arm 822 mounted to the end cap 820 and which is rotatable to move a removable needle (not shown) through patient tissue. Suturing tool 814 operates substantially the same as the first embodiment 14 with the exception of the following modifications. The needle holder arm 822 is driven by a spline gear. In an embodiment, the needle holder arm includes a drive gear 834 at a mounting end of the needle holder arm 822, and a spline gear 840 fixed at a distal end of a rotatable shaft 838. The shaft 838 is operably rotated in first and second directions by the mechanized system 51. When the shaft 838 is rotated in a first direction 854, the needle holder arm 822 is rotated into a first (open) position (FIG. 18). When the shaft is rotated in a second direction 856, the needle holder arm 822 is rotated into a second (closed) position (FIG. 19).

Turning now to FIGS. 20 and 21, a tenth embodiment of a suturing tool 914 includes an end cap 920 adapted for placement at and connection to the distal end 16 of the insertion tube 18. The end cap 920 includes a needle holder arm 922 mounted to the end cap 920 and which is rotatable to move a removable needle (not shown) through patient tissue. The needle holder arm 922 in suturing tool 914 is fixed to an axle 932 that includes a bevel driven gear 934. The bevel driven gear 934 is coupled to a bevel drive gear 940 oriented transverse to the bevel driven gear 934. The bevel drive gear 940 is coupled at the end of a rotational shaft 938 that extends a portion of the insertion tube 18. Shaft 938 is rotated by the mechanized system 51 to move the needle holder arm 922 between a first (open) position (FIG. 20), and a second (closed) position (FIG. 21).

Turning now to FIGS. 22 and 23, an eleventh embodiment of a suturing tool 1014 includes an end cap 1020 adapted for placement at and connection to the distal end 16 of the insertion tube 18. The end cap 1020 includes a needle holder arm 1022 mounted to the end cap 1020 and which is rotatable to move a removable needle (not shown) through patient tissue. The needle holder arm 1022 in suturing tool 1014 is coupled to a right-angle constant velocity joint or Hobson's Joint. More specifically, the needle holder arm 1022 is fixed relative to a right-angle rod 1040. An end 1042 of the right-angle rod 1040 is slidably positioned within an off-axis bore 1044 on a drivepiece 1046 at an end of an input shaft 1038. The input shaft 1038 is rotationally driven by the mechanized system 51. When the input shaft 1038 is driven in a first rotational direction 1050, the needle holder arm 1022 moves into a first (open) position (FIG. 22); when the input shaft 1038 is driven in a second rotational direction 1052, the needle holder arm 1022 is moved into a second (closed) position (FIG. 23).

While the above embodiments permit rotation of the needle holder arm in a plane parallel to a plane in which the longitudinal axis A_{L} of the insertion tube 18, and about an axis oriented transverse to the longitudinal axis A_{L} of the insertion tube 18 (e.g., perpendicular and/or spaced apart from), the robotic system may be adapted to move the needle holder arm and needle through space in other ways.

Turning now to FIGS. 24 through 28, a twelfth embodiment of a suturing tool 1114, preferably in the form of a replaceable module, includes a needle holder arm 1122 mounted on a shaft 1150 which is adapted to rotate the needle about a central axis A_{C} that extends parallel to, in this embodiment, coaxial with, the longitudinal axis A_{L} of the insertion tube 18. Rotation of the shaft 1150 by the mechanical drive system 51 results in rotation of the needle holder arm 1122 in a plane transverse to the longitudinal axis A_{L}, and parallel to a cross-sectional plane through the insertion tube 18. The suturing tool 1114 defines a well 1152 and a working channel 1154 extending into the well 1152. The shaft 1150 rotates the needle 26 on the needle holder arm 1122 from a first position on a first side of the well 1152 to a second position on a second side of the well. A tissue grasper such as helical grasper 70 is adapted to be longitudinally displaced relative to the working channel 1154 and retract tissue into the well 1152. A reciprocating yoke 1160 is provided at the second side of the well 1152 and adapted to engage the needle 26 mounted on the needle holder arm 1122.

Now in operation, referring first to FIG. 24, the needle holder arm 1122 is in a starting position and adapted to hold the needle 26 directed towards the well 1152, and the tissue grasper 70 and the yoke 1160 are in retracted states. Next, referring to FIG. 25, the tissue grasper 70 is longitudinally extended out of the well 1152 and rotated by the mechanized system 51 to engage tissue (not shown). Turning to FIG. 26, the tissue grasper 70 is then retracted to pull the grasped tissue into the well 1152. The needle holder arm 1122 is then rotated on shaft 1150 to advance the needle 26 through the retracted tissue. When the needle holder arm 1122 is fully advanced, the needle 26 is aligned with the retracted yoke 1160. The yoke 1160 is then advanced (in the direction of arrow 1162) to engage the needle 26. Referring to FIGS. 27 and 28, the shaft 1150 is counter-rotated in the direction of arrow 1164 to move the needle holder arm 1122 away from the engaged needle 26 and retract the needle holder arm 1122 out of the tissue. The tissue grasper 70 is released from the retracted tissue. Once the tissue moves out of the well 1152, the needle holder arm 1122 is then rotated to re-engage with the needle 26, the yoke 1160 is retracted to release the needle 26, and the needle holder arm 1122 is rotated back to the starting position so the process can be repeated.

It is further appreciated that the needle holder arm can be rotated from a shaft that extends through the tool in non-axial alignment with the longitudinal axis. By way of example, referring to FIGS. 29, 30 and 31, a thirteenth embodiment of a suturing tool 1214 substantially similar to the twelfth embodiment is shown. The shaft 1250 is located off-central axis, and causes the needle holder arm 1222 to swing outside the periphery of the insertion tube 18 when rotating, but otherwise operates in the same manner as above. The shaft 1250 is driven by the mechanized system 51.

Referring to FIGS. 32 through 36, a fourteenth embodiment of a suturing tool 1314 is shown. The suturing tool 1314 includes needle holder arm 1322 mounted to translate in a plane transverse to the longitudinal axis A_{L} of the insertion tube 18, such as in a plane parallel to a cross-section across the insertion tube 18. In some aspects, the needle holder arm 1322 includes a rack 1344 at the lower side of a drive bar 1346, and a pinion gear 1350 is provided to drive the rack 1344 in a reciprocal manner. The rack 1344 extends transverse to an upright 1345 of the arm 1322 extending parallel to the longitudinal axis A_{L} of the insertion tube 18, and the rack 1344 extends parallel to the axis A_{N} of the needle 26. The pinion gear 1350 is connected to the mechanized drive system 51, e.g., via a shaft with bevel gear or a drive belt 1338. A longitudinally displaceable yoke 1360 is provided for engaging the needle 26, to facilitate needle removal and reengagement. The tissue grasper 70 is advanceable and retractable between the needle holder arm 1322 and the yoke 1360.

In operation, the suturing tool 1314 is advanceable to a surgical site with the needle holder arm 1322 in a compact, closed position (FIG. 32). Turning to FIG. 33, once at the surgical site, the drive system 51 is operated to move the needle holder arm 1322 outward and to provide a space between the needle 26 and yoke 1360 for advancement of the tissue grasper 70. The tissue grasper 70 is longitudinally and rotationally advanced into tissue. Referring to FIG. 34, the tissue grasper 70 is then longitudinally retracted to pull tissue into the path of the needle 26 on the needle holder arm 1322. Then the needle holder arm 1322 is moved to a closed position to drive the needle 26 in a linear path through retracted tissue. The yoke 1360 is then displaced to capture the needle 26. Turning to FIG. 35, the needle holder arm 1322 is moved toward the open position and separates from the captured needle. The tissue grasper 70 is then rotated to release from the tissue. Referring to FIG. 36, the needle holder arm 1322 is moved into the closed position and reengaged with the needle 26 and the process can be repeated at another location.

Turning to FIGS. 37 through 41, a fifteenth embodiment of a suturing tool 1414, substantially similar to suturing tool 1314, is shown. The suturing tool 1414 includes two arms or jaws movable parallel to each other. A first needle holder arm 1422 moves substantially as described above, and includes a first rack 1444 provided at the top of the drive bar 1446. A second needle holder 1480 arm is provided instead of a yoke; the second needle holder arm 1480 includes a second rack 1484 provided at a bottom of its drive bar 1486. One or more pinion gears 1450 are provided to engage the first and second racks 1444, 1484 to cause relative parallel movement between the first and second needle holder arms 1422, 1480. The pinion gears 1450 are coupled to the mechanized drive system 51 (FIG. 1). The tissue grasper 70 is advanceable and retractable to draw tissue down into the linear path of the needle 26 as it is shuttled between the first and second needle holder arms 1422, 1480. In operation, the suturing tool 1414 is advanceable to a surgical site with the first and second needle holder arms 1422, 1480 in a compact, closed position (FIGS. 37 and 38). Turning to FIG. 39, once at the surgical site, the drive system 51 is operated to move the first and second needle holder arms 1422, 1480 outward from each other. The tissue grasper 70 is longitudinally advanced and rotated into tissue. The tissue grasper 70 is then longitudinally retracted to pull tissue into the path of the needle 26. Then, referring to FIG. 40, the needle holder arms 1422, 1480 are moved to a closed position to drive the needle 26 through retracted tissue and transfer engagement of the needle from the first needle holder 1422 to the second needle holder 1480. A moving yoke or other latching mechanism (not shown) may be provided to secure and release the needle in each of the first and second needle holders 1422, 1480. Turning to FIG. 41, the needle holder arms 1422, 1480 are moved into the open position and the needle 26 is transferred to the opposing arm 1480. The tissue grasper 70 is then rotated to release the tissue.

Referring now to FIGS. 42 through 45, a sixteenth embodiment of a suturing tool 1514 is shown. The suturing tool 1514 includes two jaws or arms 1522, 1580 movable in plane to pass a needle 26 through a curved path between each other. The first needle holder arm 1522 moves substantially as described with respect to the above, and includes a first rack 1544 provided along a curved first drive bar 1546. The second needle holder 1580 arm includes a second rack 1584 provided along a curved second drive bar 1586. One or more pinion gears 1550 are provided to engage the first and second racks 1544, 1584 to cause relative in-plane rotations between the first and second needle holder arms 1522, 1580. The pinion gears 1550 are coupled to the mechanized drive system 51 (FIG. 1). The tissue grasper 70 is rotatable and longitudinally displaceable to retract tissue into the path of the needle 26 between the two arms 1522, 1580. In operation, the suturing tool 1514 is advanceable to a surgical site with the first and second needle holder arms 1522, 1580 in a compact, closed position (FIG. 42). Turning to FIG. 43, once at the surgical site, the drive system 51 is operated to move the first and second needle holder arms 1522, 1580 outward relative to each other. The tissue grasper 70 is longitudinally advanced and rotated into tissue. The tissue grasper 70 is then retracted to pull tissue into the path of the needle 26. Then, referring to FIG. 44, the needle holder arms 1522, 1580 are moved to a closed position to drive the needle 26 through retracted tissue and transfer engagement of the needle from a first needle holder arm 1522 to the second needle holder arm 1580. Turning to FIG. 45, the needle holder arms 1522, 1580 are moved into the open position and the needle 26 is provided in the opposing arm 1580. The tissue grasper 70 is then rotated to release the tissue.

Referring now to FIGS. 46 through 49, a seventeenth embodiment of a suturing tool 1614 is shown. The suturing tool 1614 includes a needle holder arm 1622 movable along a curved path, preferably in substantially the same manner as the first needle holder arm 1522 of the sixteenth embodiment. The needle holder arm 1622 includes a mount 1680 to which the needle 26 can be removably connected. The mount 1680 may be curved. The tool 1614 includes a fixed needle garage 1682 adapted to receive at least a portion of the needle 26 when the needle holder arm 1622 is in a closed position, and a yoke 1660 displaceable within the garage 1682 to securely engage the needle 26. In operation, the suturing tool 1614 is advanceable to a surgical site with the needle holder arms 1622 in a closed position (FIG. 46). Turning to FIG. 47, once at the surgical site, the drive system 51 is operated to move the needle holder arm 1622 outward relative to the garage 1682. The tissue grasper 70 is longitudinally advanced and rotated into tissue. The tissue grasper 70 is then retracted to pull tissue into the path of the needle 26. Then, referring to FIG. 48, the needle holder arm 1622 is moved to a closed position to drive the needle 26 through retracted tissue. Turning to FIG. 49, the yoke 1660 is then advanced to engage the needle 26, and the needle holder arm 1622 is moved back into the open position; the needle 26 is disengaged from the needle holder arm 1622. The tissue grasper 70 is then rotated to release the tissue.

Turning now to FIGS. 50 through 53, an eighteenth embodiment of a suturing tool 1714 is shown. The suturing tool 1714 includes two needle holder arms 1722, 1780 that operates substantially similar to a dual-action claw, driven by linear actuation of coupled linkages 1750a, 1750b, 1752a, 1752b, 1754. A proximal linkage 1754 is coupled to the mechanized drive system 51 (FIG. 1). As the proximal linkage 1754 is displaced longitudinally (e.g., distally), the linkage drives the arms 1722, 1780 into an open position. As the proximal linkage 1754 is displaced longitudinally (e.g., proximally), the linkage drives arms 1722, 1780 into a closed position, and can transfer the needle 26 between the arms. As in other embodiments, the tissue grasper 70 can be advanced, engage tissue, and retracted to pull tissue into a space between the open arms, and controllably release the tissue. A similar linear actuation system can be implemented to rotate only a single arm and move the needle into a garage for engagement by a yoke, as described above.

Referring now to FIGS. 54 through 57, a nineteenth embodiment of a suturing tool 1814 is shown. The suturing tool 1814 includes a single needle holder arm 1822 that is operated by both sliding and rotating action. The tool 1814 includes a stationary forked bracket 1850 defining a split geared rack 1852 and a slot 1854 passing through the rack. The needle holder arm 1822 is rotatably mounted on a clevis 1856 at a distal end of a slide 1858. Gears 1860 are fixed relative to the needle holder arm 1822 and external of the clevis 1856. As the slide 1858 is distally displaced, the gears 1860 are rotated along the rack 1852 and cause the needle holder arm 1822 to rotate into an open position and pass through the slot 1854. As the slide 1858 is proximally displaced, the gears 1860 rotate along the rack 1852 and cause the needle holder arm 1822 to rotate into a closed position. The slide 1858 is reciprocated by the drive mechanism 51 (FIG. 1). In the closed position, the needle 26 is aligned with, and preferably seated at least partly in, a tube 1870 that is aligned with a working channel of the insertion tube, or in a needle garage, either for facilitating needle 26 removal and reengagement. The tissue grasper 70 can be advanced, engage tissue, and retracted to pull tissue into a space between the open arm 1822 and the tube 1870, and controllably release the tissue.

Turning now to FIGS. 58 through 61, a twentieth embodiment of a suturing tool 1914 is shown. The tool 1914 is adapted to rotate a curved needle 1926. The curved needle 1926 has an inner side provided with a set of gear teeth 1928. Three toothed gears 1934a, 1934b, 1934c are arranged around the periphery of the tool 1914 and positioned to ensure continual contact with the gear teeth 1928 on the needle 1926. When the gears 1934a, 1934b, 1934c are rotated about their respective axes, the needle 1926 is driven in a circular motion. At least one of the toothed gears 1934a is coupled to the mechanized drive system 51 (FIG. 1) via a rotational shaft. The toothed gears 1934a, 1934b, 1934c may be coupled together with a gear linkage, or individually driven via respective rotational shafts or motors from the drive mechanism 51. A peripheral side wall 1940 is sized to extend about the needle 1926 and gears 1934a, 1934b, 1934c to define a retracted position of the needle. The side wall 1940 includes an open portion 1942. The tissue grasper 70 is adapted to extend out of the tool 1914 (FIG. 59), engage tissue, and retract engaged tissue into tool at the open side wall portion 1942. Interior walls 1944 may be provided to guide the retracted tissue into an intended fold, in advance of piercing by the needle 1926. Referring to FIGS. 60 and 62, once the tissue is retracted and folded, the toothed gears 1934a, 1934b, 1934c are rotated to advance the actuation needle 1926 completely through the tissue. The needle 1926 may be advanced one or more times completely through the tissue. The gears may then be radially displaced inward to release the needle from the tool 1914. Other mechanisms of releasing the needle may also be used.

Turning now to FIGS. 63 through 67, a twenty-first embodiment of a suturing tool 2014 is shown. The tool 2014 is adapted to rotate a curved needle 2026 through an arc. The curved needle 2026 is mounted on an end of a curved needle holder arm 2022. The holder arm 2022 is connected to a rotational shaft 2030 via a radial arm 2032. The tool 2014 includes a magazine 2040 that stores a plurality of curved needles 2026. Each of needles 2026 preferably has an end of a length of suture (not shown) attached to the center of the needle. The magazine 2040 is mounted on struts 2042 which can be displaced to position one of the stored needles 2026 in the magazine 2040 in alignment with the end of the holder arm 2022. A displaceable yoke 2060 is provided to capture the needle 2026 attached to the end of the holder arm 2022. Referring to FIG. 63, in one manner of operation, the holder arm 2022 is provided in a ready state with the magazine 2040 arranged to position a first needle 2026 in a path of the needle holder arm. The holder arm 2022 is then rotated in a counter-clockwise first direction, and a needle 2026 from the magazine 2040 is loaded onto the holder arm 2022. Then, referring to FIG. 64, the holder arm 2022 is then rotated in an opposite clockwise second direction, shown by arrow 2050, and the magazine 2040 is retracted. Turning to FIG. 65, a tissue grasper 70 is advanced through an opening in the tool 2014, operated to engage tissue and retracted (FIG. 66) to draw tissue into the path of the first needle 2026 on the end of the holder arm 2022. Referring to FIG. 67, then the holder arm 2022 is rotated in the first direction as shown by arrow 2052 to drive the first needle 2026 through the retracted tissue and into the yoke 2060. The yoke 2060 is actuated to restrain the first needle 2026, and the needle holder arm 2022 is rotated in the second direction, shown by arrow 2054 to separate the first needle 2026 from the needle holder arm 2022. The same needle can be reloaded onto the needle holder arm for a second stitch, and then removed again. After the needle has completed its stitch, it is secured to tissue, e.g., with a knot or a suture cinch. The magazine 2040 is then advanced to position a second needle in the path of the needle holder 2022 and the process can be repeated for each of the needles in the magazine. The rotational shaft 2030, the struts 2042 attached to the magazine 2040, the yoke 2060, and the tissue grasper 70 may all be driven by the mechanized drive system 51 (FIG. 1).

Referring now to FIGS. 68 through 75, a twenty-second embodiment of a suturing tool 2114 is shown. The tool 2114 is adapted to rotate a curved needle 2126 through an arc. As shown in FIGS. 68 and 69, the curved needle 2126 is mounted on an end of a curved needle holder arm 2122. A flexible wire 2140, such as a nickel titanium wire, is attached to a proximal end 2142 of the needle holder arm 2122, and a length of suture 2128 is attached to the needle 2126. The needle holder 2122 and needle 2126 extend within a partially circular first track 2144. The first track 2144 includes a first end 2146 with a first opening 2148, and a second end 2150. The opening 2148 extends to a channel (not shown) in the insertion tube 18 (FIG. 1). A second track 2152 is provided radially internal of the first track 2144. The second track 2152 includes a first end 2154 that leads to a second opening 2156 that also communicates with the channel, and a second end 2158 that communicates with the second end 2150 of the first track via a pulley surface 2159. The second track 2152 may be serpentine, such as formed in an S-shape. The needle 2126 is positioned in the first track 2144 on one side of a well 2160. The first and second ends 2166, 2168 of the wire 2140 attached to the needle 2126 extend through the channel and are coupled to the mechanized drive system 51 (FIG. 1). At the opposite side of the well 2160, a needle yoke 2162 is provided for capturing the needle. In an embodiment, the yoke 2162 is operable by longitudinal displacement and is coupled to the mechanized drive system 51. In an embodiment, the yoke 2162 is retracted to engage the needle 2126 when the needle is located within the yoke. Alternatively, the yoke 2162 can be advanced or pivoted to capture a needle in the yoke. An opening 2164 is provided in a floor of the well 2160 for advancing the tissue grasper 70, and the tissue grasper can be advanced therethrough as previously discussed.

In an initial setup position, the first end 2166 of the wire 2140 extends rearward of the needle holder 2122, in the first track 2144, into the first opening 2148 and down through the channel. The second end 2168 of the wire 2140 extends alongside the needle holder 2122 in the first track 2144, forward of the needle 2126, out of the first track 2144, into the second track 2152, and down the second opening 2156, and into the channel. Then, the tissue grasper 70 is operated, e.g., by both rotating and advancing it (FIG. 70), to engage tissue, and then retracting the grasper to pull the tissue into the well. Next, the mechanized drive system is operated to apply sufficient tensile force to the second end 2168 of the wire 2140 to cause the wire to advance the needle holder 2122 and needle 2126 in a clockwise rotational direction about the first track 2144, and the needle 2126 to advance out of the first track and through the well 2160 (FIGS. 71 and 72). When the needle 2126 reaches the opposite end of the well 2160, the yoke 2162 is operated to capture the needle 2126 (FIG. 73). The mechanized drive system 51 is then operated to apply a sufficient tensile force on the first end 2166 of the wire to pull the needle holder 2122 in a counter-clockwise rotational direction about the first track 2144, disengaging the needle holder 2122 from the needle 2126 and retracting the needle holder 2122 from the needle 2126 (FIG. 74). The tissue grasper 70 can be disengaged from the tissue, the tool 2114 can be relocated relative to the tissue, the needle holder 2122 can again be rotated into engagement with the needle 2126, and the needle 2126 released from the yoke and returned to the starting configuration (FIG. 75) so that another suture can be made in the tissue. Other pathways and tracks for the wire 2144 can be provided.

Turning now to FIG. 76, a twenty-third embodiment of a suturing tool 2214 is shown. The tool 2214 is adapted to rotate a needle 2226. The needle 2226, with length of a suture 2328, is mounted on an end of a helical needle holder 2222 with a releasable passive latch (not shown). The needle holder 2222 is displaceable within a helical track 2244 extending about a periphery of the tool 2214. A proximal end 2230 of the needle holder 2222 is attached to an actuation arm 2232 which is coupled to the mechanized drive system 51 (FIG. 1). The drive system 51 is adapted to apply a rotational force to actuation 2232 to displace the needle holder 2222 and needle 2226 through the helical track 2244. A distal portion of the needle holder 2222 and the needle 2226 can be advanced out of helical track 2244, through a well 2260, and toward a yoke 2262 adapted to capture the needle. In an embodiment, the yoke 2262 is actuated by longitudinal displacement and is coupled to the mechanized drive system. An opening 2264 is provided in the floor of the tool for advancing a tissue grasper 70 beyond the helical needle holder 2222.

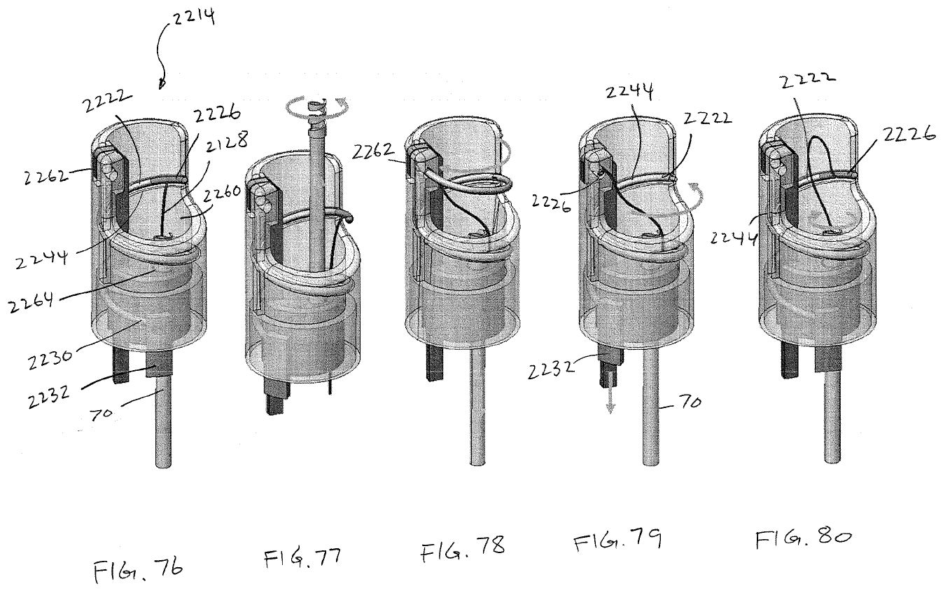

In an initial setup position, the needle holder 2222 and needle 2226 reside within the helical track 2244 and the tissue grasper 70 is in a retracted position. Then, the tissue grasper is advanced and rotated beyond the needle 2226 to facilitate engagement with tissue (FIG. 77). The tissue grasper 70 is then retracted to draw tissue into the path of the needle 2226. The actuation arm 2232 is rotationally displaced in a first direction to advance the needle holder 2222 and needle 2226 in a helical path through the retracted tissue (not shown) (FIG. 78). At the end of the needle movement, the needle 2226 extends into the yoke 2262. The yoke 2262 is then actuated to capture the needle 2226. The actuation arm 2232 is then rotationally displaced in an opposite second direction to disengage the needle holder 2222 from the needle 2226 and retract the needle holder 2222 back into the track 2244 (FIG. 79). The tissue grasper 70 then releases the tissue and the tool 2214 is moved from suture site. The needle holder 2222 is then rotationally displaced back into engagement with the needle 2226, the needle 2226 is released from the yoke 2262, and the needle holder 2222 and needle 2226 are together rotated back into the track 2244 and reset for another suture passage with tissue (FIG. 80).

Turning now to FIGS. 81 and 82, a twenty-fourth embodiment of a suturing tool 2314 is shown. The tool 2314 is adapted to rotate a needle holder 2322. A needle 2326, with length of a suture 2328, is mounted on a free end of the needle holder 2322. The needle holder 2322 is adapted to rotate about an axis 2340 oriented perpendicular to and spaced apart from the longitudinal axis A_{L} of the distal end of the insertion tube 18. Upon rotation about the axis 2340, the needle holder 2222 is displaced through a plane extending parallel to a plane in which the longitudinal axis A_{L} lies, and is adapted to pass the needle 2326 through along a path within the plane to a needle capture device, such as a secondary needle holder or a yoke 2362. A tissue grasper 70 is longitudinally displaceable through the path and adapted to draw tissue 2364 into the path so that the needle 2326 can penetrate and pass through captured and drawn tissue when the needle holder passes the needle.

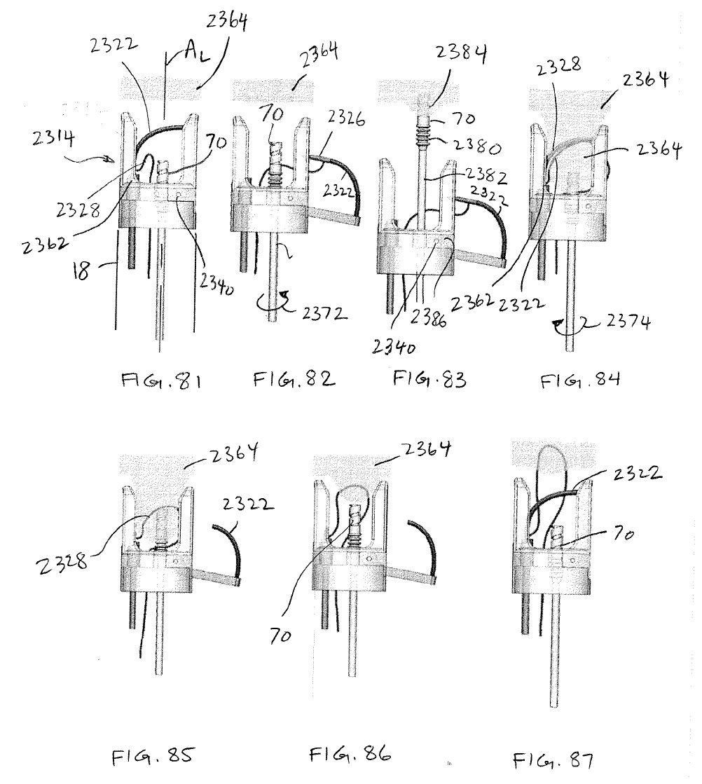

In accord with the embodiment, the tissue grasper 70 and needle holder arm 2322 are mechanically linked such that when the tissue grasper 70 is rotated in a first direction 2372 from a retracted position, the needle holder arm 2322 is moved into an open position (FIG. 82). The tissue grasper 70 can then be advanced freely of the needle holder arm 2322 and rotated into engagement with the tissue 2364 (FIG. 83). Then, the tissue grasper 70 is retracted to draw the tissue 2364 back into the path; when the tissue grasper 70 is retracted into engagement with the needle holder 2322, the tissue grasper is rotated in an opposite second direction 2374 to cause the needle holder 2322 to move into a closed position and pass the needle holder 2322, the needle 2326, and the suture 2328 through the engaged tissue 2364 (FIG. 84). The needle 2326 is engaged and retained by the yoke 2362. The tissue grasper 70 is then rotated to open the needle holder 2322 and disengage the needle holder 2322 from the needle 2326, and release engagement between the tissue grasper and the needle holder (FIG. 85). The suture 2328 extends through the tissue 2364. The tissue grasper 70 is then rotated to release the tissue 2364 (FIG. 86). The tissue grasper 70 is then retracted into engagement with the needle holder 2322 and operated to cause the needle holder to close and reengage the needle held within the yoke 2362, so that the process can be repeated as necessary (FIG. 87).

In an embodiment, the mechanical linking is a worm gear drive. A worm gear 2380 is provided to a shaft 2382 of the tissue gasper 70 proximal of the tissue grasping coil 2384 (FIG. 83). A circular gear 2386 extends about the rotational axis 2340 at the proximal end of the needle holder 2322. When the worm gear 2380 on the shaft engages the circular gear 2386 and the shaft 2382 is rotated, the needle holder 2322 rotates. The longitudinal displacement and rotation/counterrotation of the shaft of the tissue grasper can be actuated by the drive system 51.

Alternatively, the tissue grasper 70 and needle holder 2322 may be mechanically linked such that when the needle holder is actuated and moved into the open position, the tissue grasper 70 is longitudinally advanced; then, when the needle holder 2322 is moved through its path toward a closed position, the tissue grasper is retracted to pull the tissue into the path in advance of the needle.

There are various ways of removably coupling a shaft of the drive system 51 to the shaft of a tool on the suturing tool 2314 (or any of the other suturing tools described herein), such as shaft 2382 of the tissue grasper in order to provide a linear and rotational input to the tool on suturing tool. By way of example, and turning to FIGS. 88 and 89, the drive system shaft and tissue grasper shaft may be releasably coupled via a bayonet mount. In one version of such coupling, the distal end of the drive system shaft 2400 has a bayonet receiver 2402, whereas the proximal end of the tissue grasper shaft 2382 includes a crosspin 2384 (or one or two external pins) that engages in the bayonet receiver. The parts are rotated into engagement such that the drive system shaft may exert rotational and longitudinal displacement on the tissue grasper. Turning to FIGS. 90 and 91, in another example, the distal end of the driver shaft 2410 may have one lateral side with a slot 2412, and a crosspin 2404 on the input side of the tool shaft 2382 can be received in the slot. The distal end of the driver shaft 2410 can have a tapered leading end 2414 to facilitate engagement of the components.

Turning to FIG. 92, a rotary drive system 2500 can be used to selectively drive inputs to one or more tools of the suturing tool. The rotary drive system 2500 includes a central gear 2502 and a plurality of planetary gears 2504 that can be moved into and out of engagement with the central drive gear 2502 to selectively drive tools via respective drivers 2506. By way of example, engagement and disengagement can be effected by moving the individual planetary gears longitudinally, such as shown with respect to planetary gear 2504a.

There have been described and illustrated herein embodiments of robotic suturing systems for bariatric treatments, and methods of performing robotic suturing and bariatric treatments under robotic assistance. While particular embodiments of the inventions have been described, it is not intended that the inventions be limited thereto, as it is intended that the inventions be as broad in scope as the art will allow and that the specification be read likewise. Therefore, while the above systems have been described in the context of a robotic surgical system, it is recognized that the actuation systems can be used in association with manually-actuated surgical endoscopic suturing instruments. Also, the described systems can be used in conjunction with a manually actuated endoscope with a motorized surgical assist to fully or partially automate the suturing sequence. Further, while the actuation systems have been described in association with the various components of a suturing system and movement of a needle holder arm, it is appreciated that the robotic tool can otherwise be adapted to other surgical fastening tools that require similar motion to deploy a fastening element through tissue and to secure tissue. It will therefore be appreciated by those skilled in the art that yet other modifications could be made to the provided invention without deviating from its scope.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

Although embodiments of the present disclosure may be described with specific reference to medical devices and systems and procedures for endoluminal procedures, it should be appreciated that such medical devices and methods may be used with other procedures, such as percutaneous, laparoscopic, etc., procedures.

Various further benefits of the various aspects, features, components, and structures of devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A surgical system for use on a patient, comprising:
a flexible robotic instrument having a proximal end and a distal end, the distal end configured for insertion through a natural orifice of the patient to access a gastrointestinal tract of the patient; and
a fastening tool at the distal end, the fastening tool having a first fastener holder arm adapted to move a releasable tissue fastener through a path;
wherein the robotic instrument includes an actuator operably coupled to the fastener holder arm to move the first fastener holder arm, and the robotic instrument is adapted to drive the actuator to move the releasable tissue fastener from a first position at a first end of the path to a second position at a second end of the path.

2. The surgical system of claim 1, wherein the releasable tissue fastener is a suture needle coupled with a length of suture.

3. The surgical system of claim 1, wherein the actuator rotates the first fastener holder arm about an axis.

4. The surgical system of claim 1, wherein the actuator includes a flexible transmission member which is driven by being pulled into tension.

5. The surgical system of claim 4, further comprising a spring adapted to provide a counter force to the flexible transmission member.

6. The surgical system of claim 1, wherein the actuator includes a rotatable shaft.

7. The surgical system of claim 1, wherein the actuator includes a reciprocable shaft.

8. The surgical system of claim 1, wherein the actuator includes a belt.

9. The surgical system of claim 1, wherein the fastening tool has a longitudinal axis, and the first fastener holder arm is rotated in a plane about an axis oriented transverse to the longitudinal axis.

10. The surgical system of claim 1, wherein the fastening tool has a longitudinal axis, and the first fastener holder arm is rotated about a rotation axis parallel to the longitudinal axis.

11. The surgical system of claim 1, wherein the first fastener holder arm is rotated in a plane.

12. The surgical system of claim 1, wherein the first fastener holder arm is moved linearly.

13. The surgical system of claim 1, wherein the tool includes a second fastener holder arm that cooperates with the first fastener holder arm to pass the tissue fastener therebetween.

14. The surgical system of claim 1, wherein the tool includes a track, and the first holder arm is driven in the track by the actuator.

15. The surgical system of claim 14, wherein the track is partially circular or helical.

16. The surgical system of claim 1, further comprising a magazine of tissue fasteners, wherein a tissue fastener loaded on the first holder arm is deployed, a subsequent tissue fastener from the magazine can be loaded onto the first holder arm.

17. The surgical system of claim 1, further comprising a yoke operably coupleable to the robotic instrument and adapted to engage and temporarily secure the tissue fastener, wherein when the tissue fastener is secured in the yoke, the first holder arm can be decoupled from the tissue fastener.

18. A tool for a surgical system for use on a patient, comprising:
a base for coupling to a robotic surgical system;
a fastener holder arm coupled to the base and adapted to move a releasable tissue fastener through a path; and
a tissue grasper adapted to advance relative to the fastener holder arm, grasp tissue, and retract tissue into the path;
wherein the fastener holder arm and the tissue grasper are operably engaged such that, at times, movement of one of the tissue grasper and the fastener holder arm causes movement of the other of the tissue grasper and the fastener holder arm.

19. A method of operating a suturing system, the method comprising:
releasably coupling a proximal end of a suturing tool in the form of a tool module to a distal end of a flexible insertion tube of a robotic instrument having a proximal end and a distal end, the distal end configured for insertion through a natural orifice of the patient to access a gastrointestinal tract of the patient; and
operating a mechanized system to control movement of the flexible insertion tube and to operate the suturing tool.

20. The method of claim 19, further comprising converting human manual input to movement of the insertion tube via human interface controls.
